(19) United States
(12) Patent Application Publication
(10) Pub. No.: US 2020/0030273 A1
(43) Pub. Date: Jan. 30, 2020

(54) METHOD AND COMPOSITION FOR SURGERY PREPAREDNESS AND RECOVERY

(71) Applicant: Simon Frederick Harris, Burlington (CA)

(72) Inventor: Simon Frederick Harris, Burlington (CA)

(21) Appl. No.: 16/045,994

(22) Filed: Jul. 26, 2018

(51) Int. Cl. A61K 31/198; A61K 31/325; C12N 9/50; A61K 31/375; A61K 9/00

(52) U.S. Cl. CPC A61K 31/198; A61K 31/325; C12Y 304/22032; A61K 9/0053; C12N 9/63; A61K 31/375; C12Y 304/22033

(57) ABSTRACT

The present invention relates to compositions and methods to increase nitric oxide production to: accelerate wound healing, reduce complications from surgery as well as accelerating the recovery period after surgery. The compositions comprise of l-arginine and l-citrulline in ratios and dosages which enable enhanced bioavailability. The method for using the composition for promoting surgery recovery and preparedness includes the step of administering the composition to a patient pre-procedure as well as post-procedure in accordance with a prescribed protocol. The compositions may be customized to include one or more additional agents that are effective for promoting nutritional health and/or treating a particular medical problem.

### CROSS REFERENCE TO RELATED APPLICATIONS

This patent claims the benefit of U.S. Provisional Application No. 62/539,748 filed Aug. 1, 2017.

### BACKGROUND

### (i) FIELD OF THE INVENTION

The present invention relates to compositions and a method to accelerate wound healing and reduce complications and accelerate recovery associated with a surgical procedure.

### (ii) BACKGROUND

Wound healing is generally recognized to occur in three phases with different cell types performing different functions. These phases are generally divided into the inflammatory phase, followed by the proliferation phase and finally the remodeling phase.

The inflammatory phase occurs immediately after the wound is formed and can last up to 3 days. The inflammatory phase starts by blood vessel contraction and blood clot formation. Although this process is protective and stops bleeding, it can cause the tissue around the wound to become ischemic, causing an anaerobic cellular metabolism leading to acidosis, decreased cellular glycogen and ATP levels that cause reduced cell membrane function, and vascular endothelial adhesions. This leads to inflammation and complications associated with inflammation, such as pain and swelling. After bleeding stops and blood flow is restored, further damage to the ischemic tissue can be caused by free radical influx, neutrophils, and the depletion of nitric oxide. This is known as an ischemia-reperfusion injury and they can impair wound healing.

The proliferation phase occurs after the inflammatory phase and can last up to 30 days. During this phase, tissue is regenerated and blood vessels are rebuilt (angiogenesis), collagen is formed and epithelialization occurs.

Nitric oxide is a mediator in reparative processes of angiogenesis, epithelialization and collagen formation. Nitric oxide also plays a critical role in the body as a mediator of vasodilation and the immune system. Depletion of nitric oxide in the inflammatory phase causes vasoconstriction, leading to the aggravation of interstitial edema, accumulation of toxins, and the production of pro-inflammatory mediators

L-arginine is the bodies precursor to nitric oxide. Surgical trauma and pre-surgical fasting can impair l-arginine production. Orally administered l-arginine is poorly absorbed by the body due to the first metabolism by arginase and further metabolism of systemic l-arginine by the liver. This results in a negligible increase in endothelial nitric oxide production when exogenous l-arginine is administered orally.

L-arginine can be made by the body from l-citrulline. Exogenous administration of l-citrulline results in slow conversions to l-arginine due to poor extraction from systemic circulation and inefficient metabolism in the liver and digestive system.

U.S. Pat. No. 5,576,351A describes the oral administration of l-arginine and ornithine as an immunostimulator. Due to the poor bioavailability mentioned, and ornithine's lack of involvement in nitric oxide synthesis, this invention does not notably increase nitric oxide production and stimulates the immune system by inducing the secretion of pancreatic and pituitary hormones.

L-glutamine is an abundant amino acid used in protein synthesis. Exogenous l-glutamine supplementation has been reported to enhance immune response.

Proteolytic enzymes—such as bromelain—selectively catalyze the breakdown of proteins and have been shown to reduce ecchymosis and edemas.

Tissue damage, drugs and anesthetic gases used in surgery cause large amounts of physical stress on the body. Patients are left with bruising, pain, swelling and inflammation that causes pain and discomfort for up to months after the procedure, delaying their recovery and return to work and regular activities. Complications such as the formation of chronic wounds can put the patient's life at risk.

### SUMMARY

The present invention is directed to orally acceptable micronutrient compositions to enhance nitric oxide production in order to accelerate wound healing and reduce recovery time and complications associated with surgery.

The compositions essential comprise of a unit dose of l-arginine and l-citrulline in relative mass ratios between about 0.3 and 3.0 (l-arginine/l-citrulline), for a total mass of no less than 1 g and no more than 20 g.

The micronutrient compositions may further include one or more antioxidants, amino acids, proteolytic enzymes and/or hemolytic enzymes to address additional dietary and/or health concerns, and/or effects beneficial to wound healing.

The present invention is further directed to a method for administering the compositions in a timed manner to modulate the wound healing phases in order to reduce complications, accelerate wound healing, and accelerate the recovery time after surgery. The method includes the administration of said micronutrient compositions at least 2 days before a surgical procedure and continued for at least 3 days thereafter.

### DETAILED DESCRIPTION OF THE INVENTION

The present invention relates to an orally administrable composition comprising a combination of l-arginine and l-citrulline in specific ratios to enhance bioavailability and increase nitric oxide production in order to enhance wound healing and reduce complications associated with surgical trauma. The invention also may contain one or more additional antioxidants and/or amino acids used in tissue production, and/or proteolytic and/or hemolytic enzymes.

The present invention is further directed to a method for the administration of said compositions for surgical preparedness and the modulation of the phases of wound healing in order to: reduce complications from surgery (including but not limited to complications due to inflammation, in particular ischemia-reperfusion injury, pain, swelling and bruising) accelerate wound healing, as well as accelerating the recovery period after surgery.

Nitric oxide is responsible for controlling blood vessel dilation and regulating blood flow. Nitric oxide also plays a critical role in the inflammatory and proliferation phases of wound healing. Specifically, nitric oxide is a mediator in the formation of new blood vessels (angiogenesis), wound healing (epithelization) and collagen formation. Depletion of nitric oxide in the inflammatory phase is partially responsible for ischemia-reperfusion injuries, which can delay healing and lead to the formation of chronic wounds.

L-arginine and l-citrulline are used by the body in the synthesis or nitric oxide, however oral administration of l-arginine or l-citrulline on their own results in negligible increases in endothelial nitric oxide production.

L-arginine get rapidly broken down in the digestive system by the enzyme arginase as part of the urea cycle. This converts l-arginine to ornithine and urea, resulting in no nitric oxide production.

L-citrulline can be converted by the body to l-arginine. However, this process is slow and inefficient resulting in low levels of l-arginine and nitric oxide production at a given time.

The present inventions composition of orally administered l-arginine and l-citrulline taken together, in specific ratio ranges and dosages, inhibits the urea pathway and favors the production of nitric oxide. L-citrulline can inhibit arginase activity allowing more efficient absorption of l-arginine. Excess l-citrulline can be converted to l-arginine for sustained and longer acting nitric oxide production. The favoring of the nitric oxide pathway by the inhibition of arginase enables the invention to increase nitric oxide production at relatively low doses, avoiding negative side effects associated with high doses of the amino-acids which include gout, abdominal pain, acid-reflux and diarrhea.

Embodiments of the composition should contain a unit dose of at least 1 g total of l-arginine and l-citrulline in mass ratios between 0.3 and 3.0 (l-arginine/l-citrulline). The unit dose should not contain in excess of about 20 g total of l-arginine and l-citrulline as undesirable side effects such as abdominal pain and diarrhea may occur. A unit dose may contain additional antioxidants, amino acids, proteolytic and/or hemolytic enzymes. These can be used to address additional dietary and/or health concerns and/or be used to provide additional anti-inflammatory effects and/or effects beneficial to wound healing.

Examples of additional antioxidants includes, but is not limited to: resveratrol, beta-carotene, lutein, quercetin, and vitamin C.

Examples of additional beneficial amino acids includes, but is not limited to: leucine, isoleucine, valine and glutamine.

Examples of additional proteolytic and hemolytic enzymes includes, but is not limited to: bromelain and papain.

Examples of additional nutrients for a most preferred embodiment includes: vitamin C, l-glutamine, and bromelain.

Antioxidants scavenge inflammation causing free radicals which can be formed by tissue damage and oxidative stress. Vitamin C has been shown to be a powerful and well tolerated antioxidant which theoretically could help reduce inflammation,

L-glutamine is an amino acid used in protein synthesis. Exogenous l-glutamine supplementation has been reported to improve immune response.

Proteolytic enzymes (proteases) help digest the proteins in food. Exogenous supplementation of proteolytic enzymes such as bromelain, can be absorbed internally and can help reduce ecchymosis and edemas.

A unit dose of a composition in accordance with a most preferred embodiment of the present invention includes: 1 g l-arginine, 1 g l-citrulline, 2 g l-glutamine, 500 mg vitamin C and 500 mg bromelain.

In accordance with the most preferred method for surgical preparedness and recovery, one-unit dose such as that described in [032] is taken twice daily, 3 days prior to the procedure, and one-unit dose of the composition is taken twice daily for 6 days after the procedure. Other embodiments of the method may include the administration of the composition 2 to 5 days before surgery and be taken 3 days to up to 20 days after surgery.

In a preferred embodiment the composition of [032] may be individually packaged as a flavored powder in sealed foil for dilution in water and ingested orally.

In other embodiments of the invention, the composition may be packaged as a bulk powder with an appropriately sized scoop to measure out the unit dose which can then be dissolved in water. Another possible packaging option is to have the composition in a bottle or carton as an aqueous solution in either a unit dose or a bulk liquid.

It will be apparent to those skilled in the art that various other changes and modifications can be made without departing from the scope and spirit of the invention. The embodiments described do not limit the claims made to this invention and any modifications containing any or all features of the invention are intended to be covered in the appended claims.

1. A micronutrient composition to enhance nitric oxide production administered in a unit dose wherein said unit dose comprises:
a. The amino-acids l-arginine and l-citrulline in mass ratios between about 0.3 and 3.0 (l-arginine/l-citrulline) with a combined total mass of between about 1 g and 20 g.

2. A composition of claim 1, wherein said unit dose further includes one or more antioxidant(s), amino acid(s) and/or proteolytic or hemolytic enzyme(s). This includes, but is not limited to: vitamin C, glutamine, and bromelain.

3. A method to reduce complications associated with a surgical procedure comprising the step of orally administering a unit dose of the composition of claims 1 and/or 2 to the patient beginning at least 2 days prior to a surgical procedure and at least 3 days after.
